(19) United States
(12) Patent Application Publication
(10) Pub. No.: US 2005/0008535 A1
(43) Pub. Date: Jan. 13, 2005

(54) Noble gas-chlorine mixture effective against micro organisms

(76) Inventor: Alfred R. Globus, New York, NY (US)

Correspondence Address: Evelyn M. Sommer;30th Floor, 825 Third Avenue, New York, NY, 10022, US

(21) Appl. No.: 10/613,943

(22) Filed: Jul. 7, 2003

(51) Int. Cl. A61K 33/14; A61K 33/00; A01N 1/00

(52) U.S. Cl. 422/37; 424/600; 424/661

(57) ABSTRACT

A noble gas-chlorine gas mixture having broad-spectrum effectiveness against bacteria, viruses, mold, fungi, algae including spore forming microorganisms, termites, roaches, mice and other organisms. The noble gas, preferably argon/chlorine gas mixture (Chloragon) is introduced into an enclosed area where the bacteria, virus, mold, fungi, etc., are found and allowed to remain so that the gas composition can penetrate completely into the space including cracks and crevices. After the remediation is complete, the enclosed space should be ventilated or air movement increased to disperse the remaining gas composition.

### FIELD OF INVENTION

This invention relates to noble gas-chlorine gas mixtures having broad-spectrum effectiveness against bacteria, viruses, mold, fungi and algae including spore forming types and air breathing pest insects and animals, including termites, roaches, mice, rats and the like. More particularly the invention relates to a noble gas-chlorine gas mixture having a high efficacy against anthrax bacterium, Norwalk and Norwalk-like viruses, Legionellae, Severe Acute Respiratory Syndrome (SARS) virus, and black mold. The noble gas is most preferably argon.

Anthrax is an acute infectious disease caused by the spore-forming bacterium Bacillus anthracis. Anthrax most commonly occurs in wild and domestic lower vertebrates, but it can also occur in humans when they are exposed to infected animals or to tissue from infected animals or when anthrax spores are used as a bioterrorist weapon.

Anthrax infections occur in three forms cutaneous (skin), inhalation, and gastrointestinal. Most (about 95%) of anthrax infections occur when the bacterium enters a cut or abrasion on the skin, such as when handling contaminated wool, hides, leather or hair products of infected animals in a factory setting, or when handling contaminated mails or packages in post office or mail room setting. About 20% of untreated cases of cutaneous anthrax will result in death. Anthrax can be also contracted by inhalation of the anthrax spores from contaminated air and airborne anthrax infections are typically fatal. In 2001, anthrax-laden mail was sent to various government and private offices and caused nation-wide havoc and included several deaths. An efficient and low cost manner for decontaminating offices, buildings, mail-processing facilities etc., is necessary for dealing with anthrax.

Norwalk virus is the prototype strain of genetically and antigenetically diverse single stranded RNA (ribonucleic acid) viruses, formerly known as small round-structured viruses (SRSVs) that are classified in the genus Norwalk-like viruses in the family Caliciviridae. The Norwalk-like viruses can be divided into three distinct genogroups, two of which infect humans and the third that infects pigs and cows. The Caliciviridae family consists of several serologically distinct groups of viruses that have heretofore been named after the places where the outbreaks occurred.

It has been found that Norwalk and Norwalk-like viruses represent the most common cause of gastroenteritis in the United States and cause an estimated 23 million cases of acute gastroenteritis annually. Although attention has been drawn recently to outbreaks of Norwalk and Norwalk-like viruses on cruise ships, an estimated 60 to 80% of all outbreaks of acute gastroenteritis occur on land. Although many reports have focused on foodborne transmission of Norwalk and Norwalk-like viruses, recent reports highlight the potential of the viruses to cause large outbreaks in institutional settings through nonfoodborne modes of transmission. Anecdotal reports from many state health departments throughout the United States are also consistent with the recent increase in activity of the viruses within institutional and closed settings.

The cause of the increase in the viral activity is unclear, although it is probably associated with an increase in community incidence of viral infection.

Characteristics of the Norwalk and Norwalk-like viruses include a low infectious dose as few as 10 viral particles, relative stability in the environment, and their spread through multiple modes of transmission (including airborne droplets of vomitus and contact with contaminated environmental surfaces), which contributes to making Norwalk-like viruses outbreaks difficult to control.

During May-December 2002, 48 outbreaks of acute gastroenteritis were reported in the United States. Outbreak settings include restaurants and catered events, cruise ships, schools and child care centers, long term care facilities, assisted-living facilities, residential camps, sporting events, etc. Norwalk and Norwalk-like viruses were identified in 27 (73%) of the 37 outbreaks where tests were conducted.

Legionnaires' disease is a type of pneumonia that is caused by Legionella, a bacterium found primarily in warm water environments. Both the disease and the bacterium were discovered following an outbreak traced to a 1976 American Legion convention in Philadelphia.

Infection with Legionella can cause extremely serious consequences. Initial symptoms include loss of energy, headache, high fever, chest pains etc. At later stage, many bodily systems as well as the brain may be affected. The death rate ranges from about 20% for community-acquired cases to about 40% for cases acquired during a hospital stay. Long-term side effects of the disease on the survivors include fatigue and asthma.

Legionnaires' disease is contracted by inhaling airborne water droplets containing Legionellae. Water can be easily infested with Legionellae. When infested water is aerosolized, e.g. from air conditioning cooling towers, humidifiers, nebulizers, whirlpool spas, showers, the Legionellae bacterium can be dispersed, enter a person's lung and cause infection. The places of infection include homes, workplaces, hotels, hospitals, and other public places.

Severe Acute Respirator Syndrome (SARS) was first observed in late 2002 in southern China and by early 2003 has spread to multiple countries with serious social, health and economical impacts worldwide. Caused by a strain of coronavirus, SARS is transmitted to a healthy person mostly by breathing in droplets from the cough of an infected person. It can also be potentially transmitted by touching a contaminated surface, since the coronavirus has a lifetime from a few hours to 24 hours in vitro. Most of the people who have contracted the SARS disease are the relatives of the infected persons and medical personnel who have intimate contact with the infected persons in the home and hospital settings. It can also be potentially transmitted by sharing the same transportation vehicles, and by being present in other closed environments. Combining a relatively long incubation period (typically 2-7 days) and a fatality rate up to about 10%, SARS has given rise to chaos in travel, business, and of health care facilities operations with heavy financial and social consequences. Aside from isolating the infected persons, the best way to contain the spread of SARS has until now been disinfecting the air in public spaces and disinfecting object surfaces that can have been possibly contaminated.

Sachybotrys chartarum or “black mold” is widespread in the United States. Spores of the mold are carried both indoors and outdoors by air. When the spores land on to a wet surface as where the relative humidity is over 55%, the spores will be activated and grow into mold.

Black mold presents health concerns of varying degrees to the inhabitants. The EPA, in its publication titled “A Brief Guide to Mold, Moisture, and Your Home”, cautions that molds produce allergens, irritants, and in some cases potentially toxic substances (mycotoxins). Exposure by inhalation or touching of mold or mold spores can cause allergic reactions in sensitive individuals. The symptoms can include sneezing, running nose, red eyes, and skin rash (dermatitis). Molds can also trigger asthma attacks in certain individuals. In addition, mold exposure can cause irritation to the eyes, skin, nose, throat and lungs of people with both allergic and non-allergic dispositions. While the exact health concerns of mold and mold spores are still under considerable investigation and debate, certain evidence has led experts to believe that exposure to mold poses more serious health hazards than as exemplified above. The toxins, specifically mycotoxins, produced by the mold have been linked to diarrhea, headaches, fatigues, intermittent local hair loss, generalized malaise, memory loss and internal bleeding and other conditions related to mold exposure. People inhaling the toxins, especially the young and the elderly, can develop a lung disorder called pulmonary hemosiderosis, essentially a pulmonary hemorrhage. Cases have been reported in several dozens of infants who died from pulmonary hemorrhage related to mold exposure.

While the EPA guidelines provide valuable information about mold control, frequently simple drying or removal of the mold or the contaminated structure as has been proposed is not good enough or desirable. For example, some areas are hard to reach for physical removal or the item is too valuable to discard. If the mold and mold spores that are on the surface or in the air are not completely removed physically, then they will easily come back into full action when the humidity level is suitable. In some cases, even if the physical substrates are eliminated, the spores in the air remain to cause future damage.

As the microorganisms discussed above are airborne, or conveyed by droplets on contaminated surfaces, an easily dispersed and safe chemical or biological agent introduced into the environment where an outbreak has occurred or can potentially occur might be advantageous in these cases involving such places as long-term care facilities community hospitals, outpatient clinics, county jails, nursing homes, rehabilitating facilities, restaurants, schools, hotels, shelters and ships to permanently kill the microorganism and any spores formed by these microorganisms.

It has been proposed to use negative ion and ozone generators to suppress the microorganisms. The spore particles in the air are expected to carry mostly positive charges. Negative ions in the air can combine with some of the particles as result of which the particles will carry negative charges. The particles that carry opposite charges can thereafter combine and form neutral heavy particles and subsequently precipitate depositing on the ground or other surfaces. Negative ions do not kill the microorganisms. They just remove the spores from the air and deposit them on surfaces from where the spores can come back into the air at a later time. Ozone is a known strong oxidant and it has been proposed for use in effectively breaking down microorganisms in the environment by using an ozone generator. Oxygen can be converted by high voltage electricity into ozone, which can kill microorganisms by oxidizing the organic components thereof The use of ozone typically requires expensive generators to be on site due to the poor shelf lifetime of ozone. Ozone is further a toxic material and presents potential undesirable health effects.

Phenol is also a potential microorganism removal candidate since it is effective against most microorganisms. However, it is extremely toxic and highly regulated by OSHA. Its mild acidity and moderately high cost further limit its scope of application.

Much chlorine related products possessing strong oxidative properties have been tried for controlling microorganism contamination. This is sometimes effective as where the bacterial contamination is associated with the water supply. This approach is however limited and also requires installation of a chlorination system to ensure that chlorine remains in contact with water in a storage tank before the water is distributed. Chlorine bleach, whose active ingredient is hypochlorite, has been known to be relatively ineffective in fighting microorganisms, in addition to its having short shelf life stability and its hazardous effects when used directly by spraying into the environment. One successful study found that the use of chlorine dioxide, in conjunction with humidity stabilization, could effectively control microorganism in a closed environment. Chlorine dioxide is known to be a more effective biocide than phenols, bleach, glutaraldehyde, or quaternary ammonium compounds and has been widely used as an oxidizing and bleaching agent in the paper, water and food industries for many years. Low-level exposure to chlorine dioxide has very limited health effect according to the available EPA and OSHA documents. Its widespread use is limited in that it is relatively expensive and further it is explosive in concentrations in excess of 10% (v/v) at atmospheric pressure and will easily be detonated by sunlight or heat: The complexity of the handling and transformation make it unsuitable for widespread household use and also adds to the cost.

It is an object of this invention to avoid the disadvantages of the known agents for substantially eliminating microorganisms such as the Norwalk and Norwalk-like viruses, SARS virus, mold, anthrax, Legionellae and the like within institutional and closed settings.

It is a further object of the invention to eliminate the potential of outbreaks of microorganism such as the Norwalk and Norwalk-like viruses, SARS virus, mold, anthrax, Legionellae and the like in institutional and closed settings.

It is yet a further object of the invention to eliminate the source of outbreaks of disease associated with microorganisms such as the Norwalk and Norwalk-like viruses, SARS virus, mold, anthrax, Legionellae and the like particularly in institutional and closed settings including cruise ships.

Still a further object of the invention is to eliminate Legionellae, mold and mold spores found in homes, commercial buildings, HVAC systems, libraries, schools and the like by the use of mixture of chlorine gas and a noble gas.

The above objects and other advantages are accomplished by applying to the area where the microorganisms are present an effective amount of a gas composition comprising a mixture of a noble gas, preferably argon and chlorine.

### SUMMARY OF THE INVENTION

The effectiveness and safety of using chlorine in killing microbial organisms can be demonstrated by its long time use in the purification of water at parts per million levels. Obviously, a solution of chlorine is not available as a choice in treating rooms, buildings, institutional and other closed settings.

In accordance with the invention, it has been found that chlorine gas dispersed in a noble gas is effective to destroy microorganisms such as the Norwalk and Norwalk-like viruses, SARS virus and the like so that not even a background level of microorganisms, including spores associated therewith remains.

The first attempts at corrective measures were carried out with a chlorine and air mixture, containing 0.5 to 2% chlorine and it was found that the mixture could decontaminate an infected closed area in a matter of hours to overnight without the toxic, destructive effect associated with the use of chlorine itself. However, it was found that an air-chlorine mixture, standing for a long period of time, diminished in activity, presumably due to reaction by the chlorine with component(s) of air.

It was concluded that the use of an inert, non-reactive gas and namely a noble gas-chlorine mixture might be advantageously used for the desired purposes.

### DETAILED DESCRIPTION

Noble gases comprise a group of elements consisting of helium, neon, argon, krypton, xenon and radon, which show essentially no chemical reactivity under regular conditions, e.g. room temperature even in a mixture with chlorine gas. Theoretically speaking all noble gases can be used for mixing with chlorine for the microorganism control. In reality, the suitability of each noble gas is determined by its density, availability and cost as shown in Table 1.

TABLE 1
Density, abundance in air and cost of chlorine and the noble gases.

| | Density (g/cm³) at 20° C., | Abundance in air | Cost |
|---|---|---|---|
| Element | 1 atmosphere | on earth | (US$/100 g) |
| Chlorine | 0.003214 | | 0.15 |
| Helium | 0.0001787 | 0.0005% | 5.2 |
| Neon | 0.0009 | 0.0015% | 33 |
| Argon | 0.0017824 | 0.94% | 0.5 |
| Krypton | 0.003708 | 0.0001% | 33 |
| Radon | 0.00973 | | |

Radon was ruled out immediately as a candidate due to its radioactivity.

In order to make a mixture that is physically stable during storage and use, a noble gas is required that has a comparable density to chlorine. If the noble gas is too light, then it will create a blanket close to the ceiling in a closed system, preventing chlorine access to the ceiling. Likewise, if the noble gas is too heavy, then the floor will be shielded from chlorine. The cost of the noble gas is another important factor when this mixture is to be commercialized From Table 1, it can easily be appreciated that argon is the best candidate, with neon in the second place and less preferable. Argon and chlorine are the preferred mixture.

Argon and chlorine gases can be stored in separate commercial containers of appropriate material, e.g. steel tanks, equipped with calibrated dials on their release valves. When the agent is to be applied, the mixture can be generated by passing the gases from the high-pressure containers through their respective pressure regulator into a mixer. A representative design of the mixer can be a Y-shaped tube. One side is the two arms, each of which connects to one gas supply and the other side is one stem, which directs the mixed gases into the air for dispensing purpose. A representative size of the Y-shaped tube is 0.5 inch long for each of the two arms, and 1 inch long for the stem and the size can be increased or decreased according to need or convenience. The mixture can be made of any material that has sufficient strength and chemical resistance to corrosion from chlorine. A few of the exemplar materials are Monel, which is a mixture of copper and nickel; nickel itself, or stainless steel. A preferred material is Teflon (DuPont). Teflon material possesses outstanding chemical resistance to chlorine and is lightweight compared to metal or metal alloys. A suitable device, e.g. a baffle, can be installed in the stem to promote efficient mixing.

The amount of chlorine gas and the ratio of argon to chlorine can be varied according to need. For example, to clean a room 20 ft long, by 20 ft wide, by 10 ft high using the gas mixture of the invention, 20 cubic feet of chlorine, 400 cubic feet of argon, which accounts for a ratio of argon: chlorine at 20:1 (or 5% chlorine) can be utilized. Even levels of 1-3% of chlorine can work very effectively. If virus on an exposed surface is the target, as little as 0.1 to 0.2% of chlorine is sufficient.

However, it is possible to increase the level of chlorine so that the chlorine is present in an amount of up to 25% of the total mixture of chlorine and argon, especially in cases of emergency situations.

The mixture chlorine and argon (“Chloragon”) can be sprayed into the site of the microorganisms, such as bacteria, virus, spores and even where termites or mice are the problem or where black mold is present. The fact that it is in a gas form makes it possible to quickly and deeply penetrate even the smallest cracks and crevices of the enclosed area and particularly where the organism may linger to recontaminate later if a conventional method or physical removal are used. The fact that chlorine is denser than air (about 0.0012 g/cm³ at 1 atmosphere, and 20° C.) helps keep chlorine in the crevices and on top of the floor for a longer period of time for higher efficiency.

It is to be noted that the space needs to be enclosed and maintained closed for the duration of the treatment in order to maintain the concentration of chlorine substantially constant and avoid its diffusion into the atmosphere, whereby the organisms or microorganisms contained in the closed space can be destroyed. Therefore, the doors, windows and other openings need to be closed or taped shut, and the ventilation system also shut off during the application period. The length of the period can be varied depending on the target, the chlorine concentration, the volume of the mixture released, etc. In order to kill a virus exposed on a surface with Chloragon, one or two hours should be enough in most cases. While if the target is a spore former, such as anthrax one or two days may be necessary.

It may be necessary to install an alarm or detection system to detect malfunction in the chlorine-argon systems and for determining chlorine residuals

Due to the toxic effect of chlorine at relatively high concentration in the air, any person, pets, plants or other living organisms should be kept from the room or building while the site is fumigated with the Chloragon gas mixture and thorough ventilation is necessary afterwards before the inhabitants return. In typical conditions, one or two hours should be sufficient for ventilation. A fan can be used to expedite the ventilation since neither argon nor chlorine is flammable by electrical sparking.

Chlorine is a very reactive element and undergoes reaction with a wide variety of other elements and compounds. It reacts directly with most metals and many chemicals, with the exception of nitrogen, oxygen, air and carbon. Chloragon should not be applied during humid days, because the moisture can react with chlorine and produce highly corrosive HCl acid.

Argon is a noble gas, which is obtained from air is so-called inert and does not react with air, HCl, HNO₃, NaOH, etc.

The Norwalk and Norwalk-like viruses, anthrax, Legionellae, SARS virus and mold have to be attacked quickly, considering the acuteness and severity of the illnesses associated with the microorganisms in issue, and the rapid onset and communication of these illnesses. The use of the Chloragon gas compositions has the advantage that it can penetrate into the empty spaces and crevices so that the microorganisms can be eliminated completely. After the decontamination by Chloragon and before the site is reoccupied by inhabitants, the residual chlorine has to be completely removed through efficient ventilation, and the ventilation can be expedited by opening doors and/or windows, using fans or other exhaust means.

1. A composition effective against microorganisms and other pest organisms comprising a mixture of a noble gas and chlorine gas.

2. A composition according to claim/wherein said noble gas is argon.

3. A composition according to claim 1 wherein said microorganism is anthrax bacterium, Bacillus anthracis.

4. A composition according to claim 1 wherein said microorganism is Norwalk or Norwalk-like viruses.

5. A composition according to claim 1 wherein said microorganism is Severe Acute Respiratory Syndrome virus.

6. A composition according to claim 1 wherein said microorganism is Legionellae.

7. A composition according to claim 1 wherein said microorganism is mold and mold spores.

8. A composition according to claim 2 wherein said ratio of argon:chlorine in said mixture is 20:1

9. A composition according to claim 2 wherein said chlorine is present in an amount of about 0.1 to 3%

10. A composition according to claim 2 wherein said chlorine is present in an amount of about 0.1 to 5%

11. A composition according to claim 2 wherein said chlorine is present in an amount up to 25%.

12. A method for removing microorganisms which comprises introducing an effective amount of a composition according to claim 1 into the air in a room, building, ship, county jail, shelter, hospital, clinic, nursing home, rehabilitation facility, restaurant, school, hotel or HVAC system and the like, where said microorganisms are located, allowing the composition to remain for a time sufficient to substantially completely eliminate said microorganisms and then ventilating said room, building, ship, jail, shelter, hospital, clinic, nursing home, rehabilitation facility, restaurant, school, hotel or HVAC system.

13. A method for removing microorganisms which comprises introducing an effective amount of a composition according to claim 2 into the air in a room, building, ship, county jail, shelter, hospital, clinic, nursing home, rehabilitation facility, restaurant, school, hotel or HVAC system and the like, where said microorganisms are located, allowing the composition to remain for a time sufficient to substantially completely eliminate said microorganisms and then ventilating said room, building, ship or HVAC system and the like.

14. A method according to claim 12 wherein said argon and chlorine are admixed directly before their use.
